(19) United States
(12) Patent Application Publication
(10) Pub. No.: US 2026/0219179 A1
(43) Pub. Date: Jul. 30, 2026

(54) SENSING SYSTEM FOR GAS TURBINE POWER PLANT EXHAUST

(71) Applicant: Mitsubishi Power Americas, Inc., Lake Mary, FL (US)

(72) Inventors: Jesse Madsen, Longwood, FL (US); Mark Peak, Deltona, FL (US)

(21) Appl. No.: 19/036,975

(22) Filed: Jan. 24, 2025

(51) Int. Cl. G01N 21/39; F02C 6/18; G01M 15/14; G01N 33/00

(52) U.S. Cl. CPC G01N 21/39; F02C 6/18; G01M 15/14; G01N 33/0027; F05D 2220/76; G01N 2021/399

(57) ABSTRACT

A power production system comprises a gas turbine engine to combust fuel to produce gas that can produce rotational shaft power for generating electricity, an exhaust duct to receive the gas from the gas turbine engine, one or more catalyst panels positioned in a cross-sectional area of the exhaust duct to interact with the gas, an injection system to release a reductant into the gas upstream of the one or more catalyst panels, the injection system comprising a plurality of grids configured to release reductant over a portion of the cross-sectional area, and a sensing system positioned in the exhaust duct to sense composition of gas within the exhaust duct, the sensing system comprising gas composition sensors positioned in the exhaust duct in the plurality of grids to provide a gas composition reading for each of the plurality of grids.

### PRIORITY APPLICATIONS

Not Applicable

### TECHNICAL FIELD

The present disclosure is generally directed to, but not by way of limitation, gas turbine power plants that utilize an exhaust gas stream to produce rotational shaft power to produce mechanical power. More specifically, the present disclosure relates to, but not by way of limitation, systems, devices and methods for sensing composition of exhaust gas in a gas turbine power plant.

### BACKGROUND

In a gas turbine power plant, a gas turbine engine can be operated to directly generate electricity with a generator using shaft power. Compressed air and fuel can be combusted to produce exhaust gas to rotate a turbine of the gas turbine engine. The turbine can be used to drive a compressor to produce the compressed air and an electrical generator to produce electricity.

In simple cycle operation of a gas turbine engine power plant, the exhaust gas is typically vented to atmosphere, sometimes with the use of systems for removing or converting particular components within the emissions. In other configurations, gas turbine operation can be combined with a steam system. The steam systems can be used to generate steam that drives a steam turbine, which can then be used to generate electricity. Steam systems for combined-cycle power plants can typically comprise a multi-circuit heat recovery steam generator (HRSG) operating a Rankine cycle in combination with the gas turbine Brayton cycle. Working fluid for a gas turbine combined cycle (GTCC) power plant typically comprises air and/or gas (topping cycle) and steam and/or water (bottoming cycle), with a gas or liquid fuel burned in the gas turbine engine.

In order to comply with environmental regulations and other considerations, power plants can incorporate various emissions control systems to treat the exhaust gas for particular components. Gas turbine emissions can be typically treated by two systems that interact with exhaust gas of a gas turbine system. First, the exhaust gas can be passed through a catalyst system to oxidize CO from the exhaust gas into carbon dioxide (CO2), as well as oxidizing volatile organic compounds (VOCs). Second, a selective catalytic reduction (SCR) system can convert oxides of nitrogen (NOx) in the exhaust gas to nitrogen and water by a catalytic reaction of a mixture of the exhaust gas and a reducing agent or reductant, such as anhydrous ammonia, aqueous ammonia or urea. The exhaust gas and reductant mixture can react in the presence of a catalyst disposed in a panel or bed positioned in the flow path of the exhaust gas. SCR system reactors are typically placed downstream of an exhaust duct of a combustion source, such as a gas turbine or a coal boiler, and utilize the catalyst and the reductant to convert NOx into diatomic nitrogen (N2) and water (H2O).

Examples of gas turbine systems are described in U.S. Pat. No. 9,399,927 to McDeed et al., titled “Method and Apparatus for Operating a Gas Turbine Power Plant at Low Load Conditions with Stack Compliant Emissions Levels” and U.S. Pat. No. 11,286,864 to Batsch-Smith, titled “Active Inlet Turbine Control.”

### Overview

The present inventors have recognized, among other things, that a problem to be solved in emissions reduction systems is determining the effectiveness of the emissions reductions system, particularly over time. Emissions reduction systems can include the distribution of a reducing agent (reductant), such as ammonia, within an exhaust duct. It can be desirable for the reducing agent to interact with all or most portions of the exhaust gas within the exhaust duct in an equal or nearly equal manner to facilitate the reaction process with the catalyst and effectively reduce emissions. Several factors can affect the interaction of reductant with the catalyst. First, it is desirable for a sufficient level of reductant to be introduced into the exhaust duct to treat all of the exhaust gas without introducing excessive reductant in a wasteful manner. Second, it is possible for reductant to fail to make it to the desired area of the exhaust duct due to mechanical issues, such as valve or seal malfunction. Third, the catalyst bed will degrade over time and can do so in an uneven manner, thereby potentially resulting in too much reductant for the degraded portions of the catalyst bed. Thus, the present inventors have recognized that there is a need for being able to sense the composition of the materials in situ within the exhaust duct, in real time, and in different locations within the exhaust duct to evaluate and adjust emissions reduction system performance.

The present inventors have, however, recognized that it can be difficult to directly measure the contents of gas within an exhaust duct. For example, there are no known sensing devices that can directly measure gas composition in situ. Conventional exhaust duct sensing systems are temporary structures, wherein the exhaust gas is routed out the exhaust duct and into a sensing apparatus that can analyze the exhaust gas before the exhaust gas is rerouted back to the exhaust duct. Thus, such systems are ineffective at providing real time feedback concerning operation of emissions reduction systems within the exhaust duct. For example, such systems typically only sense in one location, thereby requiring additional time to take samples at multiple locations of the exhaust duct.

The present subject matter can help provide solutions to these problems and other problems, such as by providing exhaust gas measurement systems, devices and methods that allow for real-time assessment of exhaust gas composition, including at different locations within the cross-sectional area of an exhaust duct and at different axial positions along the exhaust duct. In examples, a plurality of sensing devices, such as tunable diode laser devices, can be positioned alongside the exhaust duct to emit lasers therein. Each sensing device can emit multiple laser beams to be able to obtain baseline readings and sample readings at precise locations within the exhaust duct. Real time data from sensing devices can be used to troubleshoot operation of emissions reduction systems. In particular, data from the sensing devices can be used to determine over or under application of the reducing agent and degradation of the catalyst bed, among other things. Thereafter, an automated valve system can be automatically operated by a computer control system to adjust flow of injected reductant to, for example, match reductant supply with reductant demand. Furthermore, feedback from the sensing devices can be used to troubleshoot aspects of the remissions reduction system, such as to facilitate the determination of a degraded catalyst, the locations of leaks and other potential issues.

In an example, a power production system can comprise a gas turbine engine configured to combust a fuel to produce a gas that can be used to produce rotational shaft power for generating electricity, an exhaust duct configured to receive the gas from the gas turbine engine, the exhaust duct having a cross-sectional area, one or more catalyst panels positioned in the exhaust duct to substantially cover the cross-sectional area and interact with the gas, an injection system configured to release a reductant into the gas upstream of the one or more catalyst panels, the injection system comprising a plurality of grids, each grid of the plurality of grids configured to release reductant over a portion of the cross-sectional area, and a sensing system positioned in the exhaust duct to sense composition of gas within the exhaust duct, the sensing system comprising a first plurality of gas composition sensors positioned in the exhaust duct, wherein each of the first plurality of gas composition sensors is paired with one of the plurality of grids of the injection system to provide a gas composition reading for each of the plurality of grids.

In another example, a gas composition sensor for use in an exhaust duct of a gas turbine power plant can comprise a first pipe comprising a first end portion connected to the exhaust duct and a second end portion positioned within the exhaust duct at a location where gas composition is to be sensed, a transmitter configured to emit a first laser beam to sense a first gas composition within the first pipe and a second laser beam to sense gas composition in the exhaust duct outside of the second end portion, a receiver configured to receive the first laser beam and the second laser beam to determine a first composition signal and a second composition signal, and a controller configured to subtract the first composition signal from the second composition signal to determine gas composition at the location.

In an additional example, a method of determining composition of exhaust gas in a gas turbine power plant having an emissions reduction system configured to dispense reductant onto a catalyst bed can comprise emitting a first laser beam into a first sensing tube extending into an exhaust duct, emitting a second laser beam into the first sensing tube and into the exhaust duct, sensing a first reductant level in the first sensing tube using the first laser beam, sensing a second reductant level in the exhaust duct using the second laser beam, subtracting the first reductant level from the second reductant level to determine a first reductant value, performing a comparison of the first reductant value to an expected reductant value, and adjusting a reductant valve to adjust an amount of reductant dispensed onto the catalyst bed based on the comparison.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

FIG. 1 is a cross-sectional view of a gas turbine system in which an exhaust gas sensing system of the present disclosure is used.

FIG. 2 is a cross-sectional view of a power plant including an emissions reduction system and an exhaust gas sensing system incorporated into an exhaust duct.

FIG. 3 is a schematic front view of an injection grid for an emissions reduction system positioned relative to an exhaust duct.

FIG. 4 is a schematic perspective view of an exhaust gas measurement system of the present disclosure placed within the exhaust duct of FIG. 3 along with the injection grid and a catalyst bed.

FIG. 5 is a schematic cross-sectional view of an exhaust gas sensing apparatus of the present disclosure comprising a tunable diode laser system.

FIG. 6 is a block diagram of methods for sensing gas composition of exhausts gas, operating an exhaust gas sensing system and adjusting operation of an emissions reduction system according to the present disclosure.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

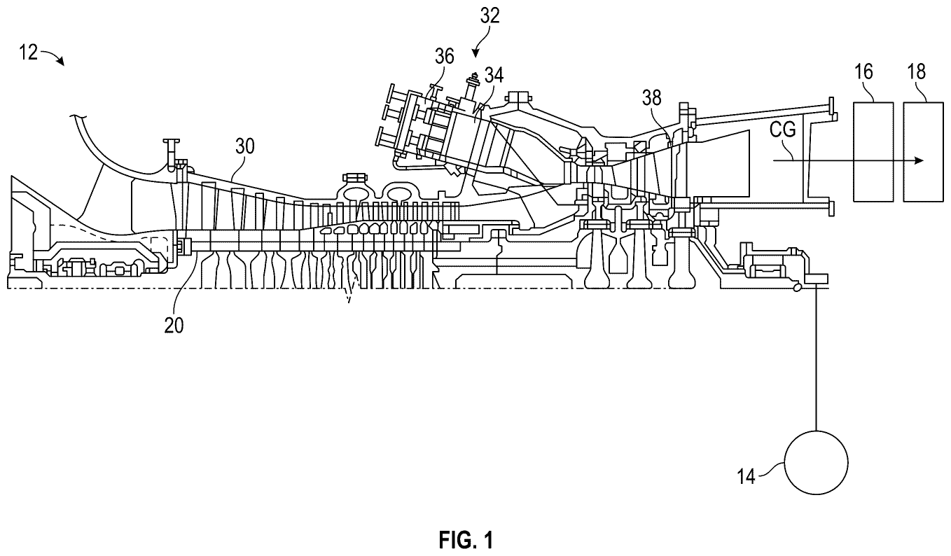

FIG. 1 is a cross-sectional view of an embodiment of gas turbine system 12 used to provide rotational input to generator 14. Combustion gas CG from gas turbine system 12 can be routed through emissions reduction system 16 and sensing system 18 of the present disclosure. Gas turbine system 12 can comprise compressor 30 and combustor 32. Combustor 32 can include combustion region 34 and fuel nozzle assembly 36. Gas turbine system 12 can also include gas turbine 38, which can be coupled to compressor 30 via common shaft 20. In operation, air can enter the inlet of compressor 30, can be compressed and then discharged to combustor 32 where fuel, such as a gas, e.g., natural gas, or a fluid, e.g., oil, injected from fuel nozzle assembly 36 is burned to provide high energy combustion gases that can drive gas turbine 38. In gas turbine 38, the energy of the hot gases is converted into work at common shaft 20. Common shaft 20 can be used to drive compressor 30 and other loads such as generator 14 to produce electricity.

Combustion gas CG exiting gas turbine system 12 can be exhausted to atmosphere, such as through an exhaust duct and a stack, or can be converted into additional useful work, such as in a heat recovery steam generator (HRSG), before being exhausted to atmosphere. In additional examples, exhaust gas can be generated by other types of combustion processes, including coal plants and the like. Emissions reduction system 16 can be used to remove harmful or potentially harmful constituents of combustion gas CG, whether used with gas turbine system 12 or other combustion gas generation systems.

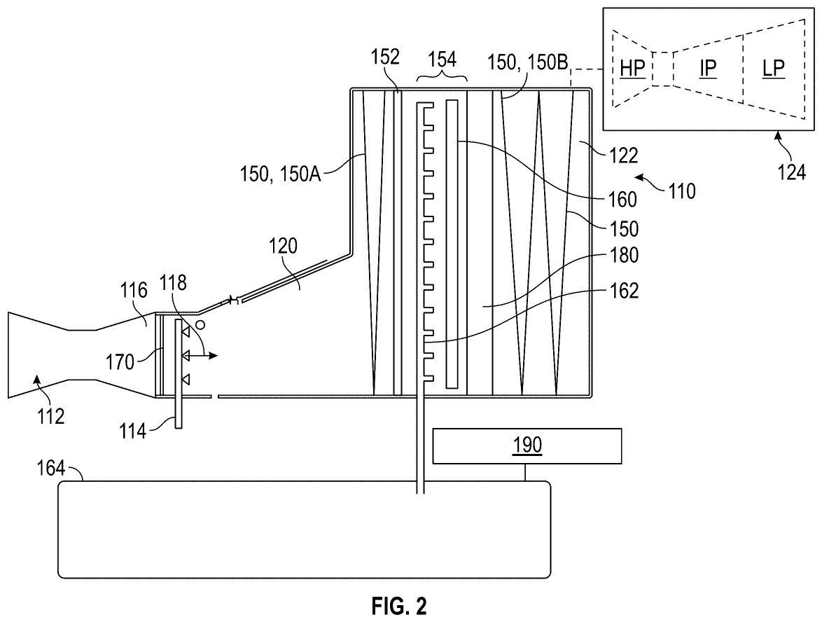

Governmental agencies have required power plants to meet environmental emissions limits. For example, environmental emissions limits can set maximum emissions for nitrogen dioxide (NOx) and carbon monoxide (CO) during various operating conditions of the power plant, such as at start-up and full capacity. As such, power plants can include various forms of emissions reductions systems. For example, combustion gas CG can be passed through emissions reduction system 16, which can comprise a CO catalyst system within an exhaust duct to oxidize CO to CO2. Emissions reduction system 16 can also be configured to oxidize VOCs. Further, emissions reduction system 16 can include a selective catalytic reduction (SCR) system within the exhaust duct or HRSG that can convert NOx to nitrogen and water by causing a mixture of a reducing agent, e.g., anhydrous ammonia, aqueous ammonia or urea, and the exhaust to react in presence of a catalyst that facilitates the reaction. Examples of SCR systems that can comprise emissions reduction system 16 are discussed in greater detail with reference to FIG. 2.

With the present disclosure, sensing system 18 can be used to sense the composition of combustion gas CG before, during or after passing through emissions reduction system 16 to evaluate and adjust the effectiveness of emissions reduction system 16. Sensing system 18 can provide two-dimensional (e.g., at different locations within a cross-sectional area) and three-dimensional (e.g., at different axial positions) gas composition readings of combustion gas CG within an exhaust duct to allow for pinpoint determinations of exhaust gas composition. As such, the effectiveness of emissions reduction system 16 at different locations within the cross-sectional area and along the length of the exhaust duct can be determined. In response, emissions reduction system 16 can be adjusted to improve the effectiveness of emissions reduction system 16 and reduce waste. In examples, emissions reduction system 16 and sensing system 18 can be operated automatically in conjunction with each other, such as via a computer operated control system, to obtain emissions readings from sensing system 18 and adjust components of emissions reduction system 16, such as reducing agent injection valves. Though the present application is described with reference to sensing system 18 being used to analyze the reducing agent component of combustion gas CG, sensing system 18 can be used to sense for other materials, gases, constituents and the like. For example, sensing system 18 can be used to sense moisture composition within combustion gas CG, which can be useful when hydrogen gas is being burned in combustor 32.

FIG. 2 is a cross-sectional view of power plant 110 including gas turbine system 112 operatively coupled to exhaust duct 122. Gas turbine system 112 can include one or more of any conventional combustion-based gas turbine engines, such as the one shown in FIG. 1. Gas turbine system 112 can optionally include duct burner 114 downstream of gas turbine 116, which can burn additional fuel to raise the temperature of exhaust gas 118 exiting gas turbine 116 in a conventional manner. Exhaust gas 118 can include a variety of combustion byproducts such as carbon dioxide, carbon monoxide (CO), nitrogen oxide (NOx), Volatile Organic Compounds (VOCs) and the like. Exhaust gas 118 can pass through exhaust passage 120 operatively coupled to gas turbine 116 and exhaust duct 122. Exhaust passage 120 can be configured to direct exhaust gas 118 downstream of gas turbine 116, such as to exhaust duct 122. Exhaust passage 120 can be an integral part of exhaust duct 122, or can be a separate passage upstream but operatively coupled to exhaust duct 122. Exhaust passage 120 defines a portion of an exhaust path for exhaust gas 118 that continues into exhaust duct 122.

In examples, power plant 110 can comprise a simple cycle power plant where exhaust gas of gas turbine system 112 is vented to atmosphere after passing through exhaust duct 122. In examples, exhaust duct 122 can be operably coupled to exhaust passage 120 of gas turbine 116 for generating steam for steam turbine 124, which is shown schematically in phantom in FIG. 2. In such configurations, exhaust duct 122 can include a steam generating heat exchanger and can include heat exchange pipes 150 through which water and/or steam can be passed to increase the temperature and energy level of the water and/or steam. For example, exhaust duct 122 can include heat exchange pipes 150 that can function as conventional parts of a HRSG such as but not limited to: superheater(s), economizer(s) and reheat section(s) for any number of steam turbine stages (i.e., HP, IP and/or LP). Any conventional steam or boiler drums (not shown) can also be provided as part of a HRSG in conjunction with exhaust duct 122 to be fluidly connected to the steam system. A HRSG can also include various piping or valving (not shown) to deliver water/steam, as desired. However, when exhaust duct 122 is configured for simple cycle operation, heat exchange pipes 150 can be omitted.

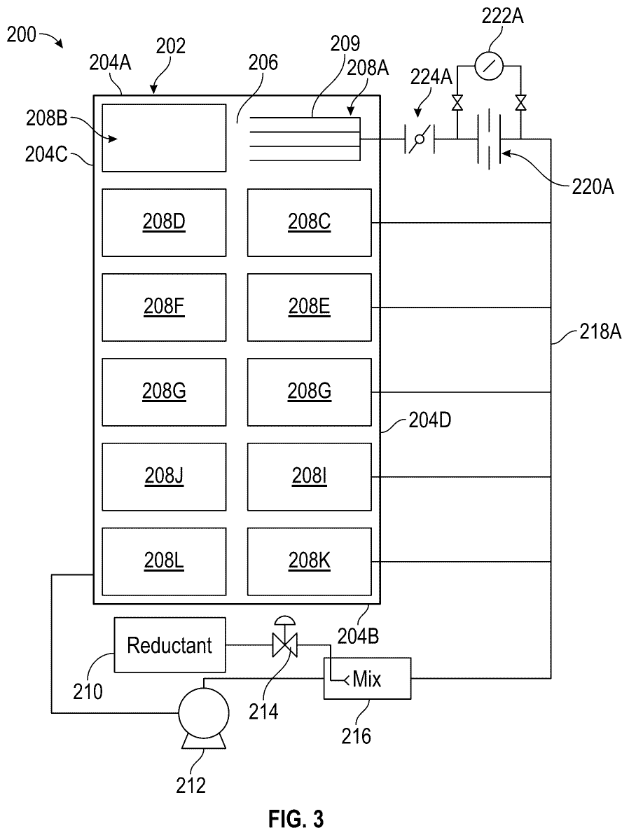

Exhaust duct 122 can also include a conventional carbon monoxide (CO) catalyst 152 downstream of a first set of heat exchange pipes 150A. Catalyst 152 can include a CO catalytic material capable of carrying out the desired catalytic conversion of CO to carbon dioxide (CO2) or other less toxic pollutants in a conventional manner. Exhaust duct 122 can also include a selective catalytic reduction system (SCR), e.g., SCR system 154, which can be located upstream of a second set of heat exchange pipes 150B. SCR system 154 can include SCR catalyst 160 and an SCR reducing agent injector 162. SCR system 154 can convert NOx to nitrogen and water by causing a mixture of the exhaust gas and a reducing agent, as provided by reducing agent injector 162, to react in presence of SCR catalyst 160. Reducing agent injector 162 can inject a reducing agent such as anhydrous ammonia, aqueous ammonia or urea, and SCR catalyst 160 can comprise a panel formed of or that can include a porous catalyst material, such as a metal oxide or zeolite based porous catalyst. Reducing agent injector 162 can comprise an ammonia injection grid (AIG), such as injection panel 200 of FIG. 3.

In examples, SCR catalyst 160 can take the form of combined SCR/CO catalyst. In such a configuration, SCR catalyst 160 can include both SCR layers and CO catalyst layers, and is functional to remove both NOx and CO.

SCR reducing agent injector 162 can be coupled to any form of reductant delivery system 164 for delivery of a reducing agent, such as to entrain the reducing agent within a flow of air, such as exhaust gas 118 for example. Reductant delivery system 164 can comprise appropriate tanks, pumps, pipes, valves and the like to deliver reductant to SCR reducing agent injector 162. Controller 190 can be employed to control the afore-described components. Controller 190 can be configured, e.g., via hardware and/or software modifications, adjustments, alterations and/or operations, to control various operational functions of reductant delivery system 164 which deliver reducing agent to SCR reducing agent injector 162. In particular, controller 190 can be configured to operate reductant delivery system 164 based on output of gas sensing system 180, described below.

SCR catalyst 160 can span, or substantially span, the width and height of exhaust duct 122. SCR reducing agent injector 162 can comprise an injector system such as an array of nozzles, sprayers, etc., capable of mixing the reducing agent with exhaust gas 118 and supplying such mixture to SCR catalyst 160. SCR reducing agent injector 162 can be configured to introduce, e.g., inject, reductant, across the width and height of exhaust duct 122. In examples, SCR reducing agent injector 162 can be permanently mounted within exhaust duct 122, or can be configured as an add-on system. SCR reducing agent injector 162 can include metal piping and nozzles capable of withstanding the temperatures and load placed on it by exhaust gas 118 of gas turbine 116. For the avoidance of doubt, as used herein, the term “exhaust duct” shall mean any structure into which a catalyst 152 or SCR system 154 may be disposed, such that the exhaust duct shall force the flow of exhaust gases through the catalyst 152 and/or SCR system 154. Although depicted in FIG. 2 as part of a HRSG structure, the scope of the disclosure is not so limited, and exemplary exhaust ducts into which a catalyst 152 or SCR system 154 may be disposed may be part of a thermal power plant, such as simple cycle gas turbine power plant or coal power plant, for example.

In operation, the reducing agent is injected into exhaust gas 118 upstream of SCR catalyst 160 via SCR reducing agent injector 162. Thus, exhaust gas 118 passes through SCR reducing agent injector 162 mixing with reductant and then continues through the SCR catalyst 160. As the mixture of exhaust gas 118 and reductant pass through SCR catalyst 160, the NOx and the reductant within the mixture react in the presence of SCR catalyst 160, which, via catalytic reduction, reduces NOx to nitrogen and water, which then may be exhausted to atmosphere.

Exhaust duct 122 can also include flow distributor 170 upstream of duct burner 114 to distribute the exhaust gas flow within exhaust passage 120 in case the profile of mass and/or velocity of the exhaust gas 118 is not uniform, such as can arise during certain operating conditions of gas turbine 116, e.g., during start-up. Flow distributor 170 can include a perforated plate or some other design to distribute the flow properly, e.g., uniformly. Likewise, another flow distributor similar to flow distributor 170 but larger in area, can be placed upstream of catalyst 152 or SCR system 154 to distribute exhaust gas within exhaust duct 122 upstream of SCR reducing agent injector 162.

SCR reducing agent injector 162 can comprise a reductant distribution system within the exhaust stream having a plurality of distribution grids. See grid 208A of FIG. 3 as an example distribution grid. The reductant can be injected into the exhaust stream from a plurality of distribution branches (e.g., distribution branch 209 of FIG. 3) or injection lances forming each distribution grid. Each distribution grid can be connected to a common supply manifold. The distribution branches are typically oriented at approximately ninety degrees to the supply manifold and run an entire width (or height) of the exhaust duct into which the SCR catalyst is disposed. Each distribution branch can be fed at one end by the supply manifold and can have a closed opposite end. The distribution branches have a plurality of openings or orifices from which the reductant is injected into the exhaust stream.

Each distribution grid can be positioned to apply reductant to a portion of the cross-sectional area of SCR catalyst 160 (as extending into the plane of FIG. 2). In prior art systems, each distribution grid is pre-tuned to deliver an appropriate amount of reductant to SCR catalyst 160 given particular volumetric exhaust gas flow within the exhaust duct at that location for one operating state of the gas turbine engine. However, the volumetric exhaust gas flow within the exhaust duct can change in a different manner in each location for different operating states of the gas turbine engine. Furthermore, SCR catalyst 160 will degrade over time at varying rates in different locations. As such the amount of reductant desirable at each two-dimensional position of SCR catalyst 160 can vary based on the operating state of the gas turbine engine and associated volumetric flow rates, and the condition of SCR catalyst 160 at that location. With the present disclosure, the amount of reductant delivered at each grid can be tuned in real time for the current operating state of the gas turbine engine and the associated volumetric exhaust gas flow within the exhaust duct at that location, as well as to take into account different states of SCR catalyst 160, among other reasons.

Gas sensing system 180 can comprise a gas composition sensing system that includes a plurality of sensing devices to sense the gas composition of exhaust gas on either side of SCR catalyst 160, thereby facilitating sensing of reducing agent injected into SCR catalyst 160 and reducing agent consumed by SCR catalyst 160. Furthermore, gas sensing system 180 can include a plurality of sensing devices distributed over the cross-sectional area of SCR catalyst 160 to sense in different grid zones. As such, gas sensing system 180 can provide inputs to SCR system 154 to customize the output of SCR reducing agent injector 162 to match operating conditions and the state of SCR catalyst 160, thereby reducing waste of reducing agent and helping to avoid under-injection of reducing agent.

FIG. 3 is a schematic front view of injection panel 200 positioned relative to exhaust duct 202. Exhaust duct 202 can comprise upper panel 204A, lower panel 204B, left panel 204C and right panel 204D. The four panels 204A, 204B, 204C, and 204D can form flow path 206. Injection panel 200 can comprise a plurality of injection grids, including grid 208A. Grid 208A can include a plurality of distribution branches, such as distribution branch 209. Injection panel 200 can also comprise grid 208B, grid 208C, grid 208D, grid 208E grid 208F, grid 208G, grid 208H, grid 208I, grid 208J, grid 208K and grid 208L. In the illustrated example, injection panel 200 can comprise twelve injection grids, each including a plurality of distribution branches. In examples, injection panel 200 can comprise SCR reducing agent injector 162 of FIG. 2.

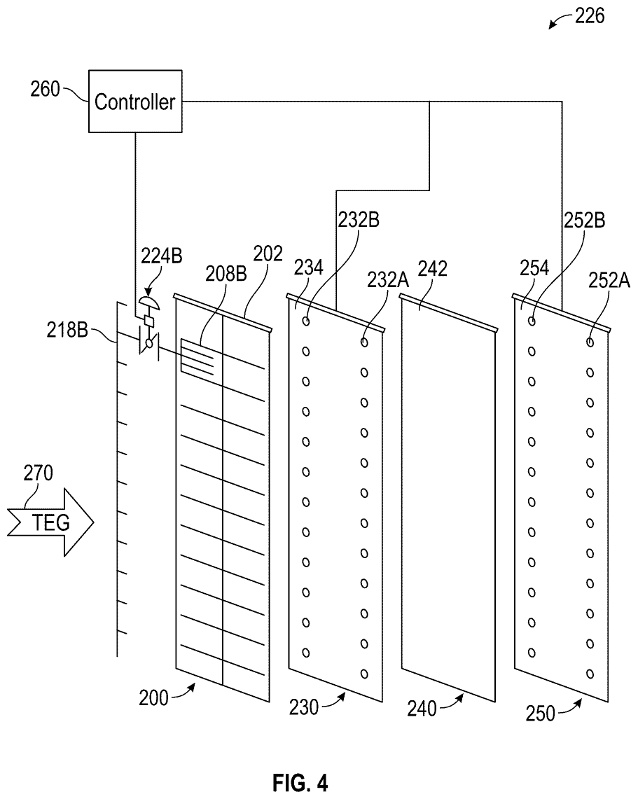

Injection panel 200 can further comprise or be connected to reductant source 210, fan 212, inlet valve 214, mix chamber 216, manifold 218A, orifice 220A, pressure gauge 222A and valve 224A. A second manifold can be used to introduce reductant into the left side of exhaust duct 202 from mix chamber 216. Thus, manifold 218A can provide reductant to grid 208A, grid 208C, grid 208E, grid 208G, grid 208I and grid 208K, and another manifold that is a mirror image of manifold 218A can provide reductant to grid 208B, grid 208D, grid 208F, grid 208H, grid 208J and grid 208L. Orifice 220A, pressure gauge 222A and valve 224A can be used in conjunction with grid 208A. Each of grid 208B through grid 208L can additionally include an orifice, a pressure gauge and a valve, though not illustrated for simplicity, to receive reducing agent from mix chamber 216. Thus, each of grid 208A through grid 208L can have a dedicated valve to control the amount of reducing agent from mix chamber 216 that reaches each grid. In the illustrated example, exhaust duct 202 comprises a rectangle. Each grid, e.g., grid 208A, grid 208B, etc., can comprise a rectangular group of distribution branches that can cover a portion of the cross-sectional area of exhaust duct 202. As shown in FIG. 4, catalyst bed 240 can be positioned within exhaust duct 202 behind grid 208A through grid 208L. Thus, one valve can control reducing agent flow from one of the grids into a portion of the catalyst bed behind, e.g., downstream of the catalyst bed.

During operation, combustion gases can be directed into exhaust duct 202, into the plane of FIG. 3. A reducing agent from reductant source 210 can be introduced into the right side of exhaust duct 202 via manifold 218A and the left side of exhaust duct 202 via a corresponding manifold (not illustrated). Fan 212 can be used to provide dilution gas (typically either hot gas drawn from within the duct or ambient air) into mix chamber 216. The reducing agent from reductant source 210 can be mixed with dilution air before being injected into exhaust duct 202 to help ensure distribution and prevent localized high concentrations of the reducing agent. In the illustrated example, the reducing agent comprises ammonia. In examples, ammonia gas can be provided from reductant source 210 to mix chamber 216. Inlet valve 214 can control the amount of ammonia provided to mix chamber 216. In examples, the reducing agent and dilution gas can be mixed to comprise 19% reducing agent, e.g., ammonia. From mix chamber 216, the diluted reducing agent can flow to manifold 218A and another manifold on the opposite side of exhaust duct 202. Fan 212 can provide the motive force to the diluted reducing agent for flow through manifold 1218A.

Flow of diluted reductant from each manifold into each of the grids can be controlled by a valve. For example, flow from manifold 218A to grid 208A can be controlled by valve 224A. Likewise, flow into each of grid 208B, grid 208C, grid 208D, etc. can be controlled by a valve. Flow of diluted reductant into each valve can be preceded by flow through an orifice, such as orifice 220A. Orifice 220A can be used in conjunction with pressure gauge 222A to obtain an indication of the amount of diluted reductant flowing into grid 208A. Again, flow into each of grid 208B, grid 208C, grid 208D, etc. can be measured with a corresponding pressure gauge.

In prior art configurations, flow into grid 208A can be determined via output of pressure gauge 222A. Valve 224A can then be manually adjusted to increase or decrease the amount of reducing agent flowing into grid 208A to meet the needs of the catalyst located downstream of grid 208A. Typically, valve 224A and the other corresponding valves are set only during initial configuration of injection panel 200 and therefore are tuned for only one operating state of the system. Furthermore, pressure gauge 222A can only provide an indication of volumetric flow of the diluted reducing agent entering into exhaust duct 202 and cannot provide a real-time indication of the amount of reductant within exhaust duct 202, such as downstream of injection panel 200 or the catalyst bed. Thus, prior art systems do not provide a high level of control over the amount of flow of reducing agent into exhaust duct 202. Furthermore, prior art systems do not provide any indication of how much reducing agent is being consumed within exhaust duct 202.

With the present disclosure, a sensing system, e.g., measurement system 226 of FIG. 4, can be included within exhaust duct 202 behind or downstream of injection panel 200. In examples, one or more sensing arrangements can be included to sense the composition of exhaust gas upstream and downstream of a catalyst bed to determine how much reducing agent is put into the system and how much reducing agent is leaving the system. With the present disclosure, use of manually operated valves, such as valve 224A, can be replaced by automated valves, such as valve 224B of FIG. 4, to allow for automated adjustment of reducing agent injection to account for changes in operating conditions of gas turbine system 112 (FIG. 2), variations in catalyst bed 240, as well as other variables.

FIG. 4 is a schematic perspective view of measurement system 226 of the present disclosure that can be placed within exhaust duct 202 of FIG. 3. Measurement system 226 can comprise first sensing panel 230 and second sensing panel 250, between which can be located catalyst bed 240. First sensing panel 230, second sensing panel 250 and valve 224B can be connected to controller 260. Turbine exhaust gas (TEG), such as TEG 270, can flow through injection panel 200, first sensing panel 230, catalyst bed 240 and second sensing panel 250. Controller 260 can comprise controller 190 of FIG. 2 and measurement system 226 can comprise gas sensing system 180 of FIG. 2.

Reductant can be delivered to grid 208B via manifold 218B from mix chamber 216. With the configuration of FIG. 4, manifold 218B can be connected to grid 208B via valve 224B. The right side of exhaust duct 202 can additionally be provided with a manifold, such as manifold 218A of FIG. 3. However, in the embodiment of FIG. 4, pressure gauge 222A and orifice 220A can be omitted, but can be included is desired. Furthermore, with the embodiment of FIG. 4, valve 224A and valve 224B can comprise automated valves, rather than manually operated valves. Only grid 208B and valve 224B are illustrated in FIG. 4, but injection panel 200 can include a plurality of grids as shown in FIG. 3, each grid having an automated valve that can be adjusted by controller 260 based upon, for example, output of first sensing panel 230 and/or second sensing panel 250.

First sensing panel 230 can comprise a plurality of sensing zones, such as sensing zone 232A and sensing zone 232B. Second sensing panel 250 can comprise a plurality of sensing zones, such as sensing zone 252A and sensing zone 252B. First sensing panel 230 and second sensing panel 250 can comprise a commensurate number of sensing zones as there are grids within injection panel 200. Thus, for the illustrated example, first sensing panel 230 and second sensing panel 250 can include twelve sensing zones arranged in a 2×6 configuration.

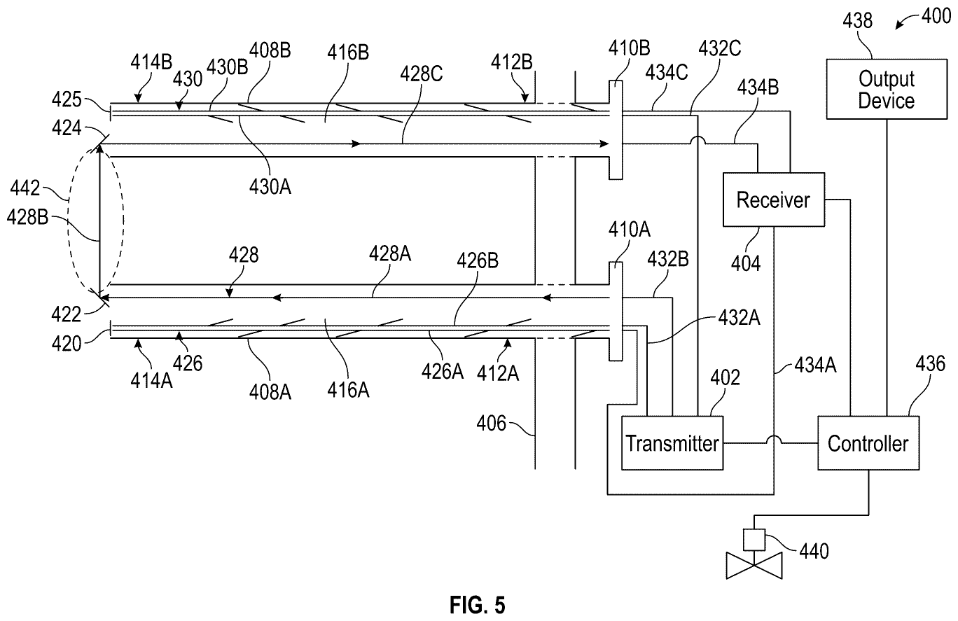

Sensing zone 232A and sensing zone 232B can comprise sensors, apparatuses or components for transmitting sensor signals, such as laser signals. In examples, each of the sensing zones of first sensing panel 230 can comprise an instance of sensing zone 442 of FIG. 5. Sensing zone 232A and sensing zone 232B can be mounted within exhaust duct 202 via framework 234. First sensing panel 230 can be used to sense reducing agent emitted from injection panel 200 within TEG 270, before entering catalyst bed 240.

Catalyst bed 240 can comprise a body of material of a catalyst. Catalyst bed 240 can be mounted in exhaust duct 202 via framework 242. Catalyst bed 240 can be configured to interact with reducing agent from injection panel 200 to remove emissions from TEG 270. Catalyst bed 240 can be porous to allow TEG 270 to pass therethrough.

Sensing zone 252A and sensing zone 252B can comprise sensors, apparatuses or components for transmitting sensor signals, such as laser signals. In examples, each of the sensing zones of second sensing panel 250 can comprise an instance of sensing zone 442 of FIG. 5. Sensing zone 252A and sensing zone 252B can be mounted within exhaust duct 202 via framework 254. Second sensing panel 250 can be used to sense reducing agent within TEG 270 after having passed through catalyst bed 240.

In examples, injection panel 200 can be used with one or both of first sensing panel 230 and second sensing panel 250. In an example, only second sensing panel 250 can be used, particularly when sensing for reducing agent composition and effectiveness of catalyst bed 240. However, only first sensing panel 230 can be used if it is desired to determine the composition of TEG 270 for other constituent gases than reducing agent, such as moisture or to confirm the uniformity of reducing agent within the exhaust duct upstream of the catalyst bed 240.

First sensing panel 230, catalyst bed 240 and second sensing panel 250 can have shapes configured to align with injection panel 200. In the illustrated example, framework 234, framework 242 and framework 254 can be rectangular shaped to match with the cross-sectional shape of exhaust duct 202. Panels 204A, 204B, 204C and 204D can extend axially across first sensing panel 230, catalyst bed 240 and second sensing panel 250 to form a rectangular enclosure, but are omitted from FIG. 4 for clarity.

First sensing panel 230, catalyst bed 240 and second sensing panel 250 can be axially aligned with injection panel 200. First sensing panel 230 can be positioned downstream of injection panel 200. Catalyst bed 240 can be positioned downstream of first sensing panel 230. Second sensing panel 250 can be positioned downstream of catalyst bed 240. Thus, flow of TEG 270 can flow sequentially through injection panel 200, first sensing panel 230, catalyst bed 240 and second sensing panel 250. First sensing panel 230 and second sensing panel 250 can be positioned in close proximity to catalyst bed 240. For example, it is desirable for first sensing panel 230 to be sufficiently downstream from injection panel 200 to allow reducing agent released therefrom to diffuse into TEG 270. Likewise, it can be desirable to sense with second sensing panel 250 in close proximity to catalyst bed 240 to prevent intermixing of TEG 270 from adjacent zones.

During use, TEG 270 can flow through exhaust duct 202 (FIG. 3). Injection panel 200 can be used to introduce a reducing agent into exhaust duct 202 that is desired to cover all or most of the cross-sectional area of exhaust duct 202. First sensing panel 230 can be used to measure the composition of TEG 270 between injection panel 200 and catalyst bed 240 to evaluate the effective spread of reducing agent across the cross-sectional area of exhaust duct 202. Intermixed TEG 270 and reducing agent can flow into and through catalyst bed 240. Within catalyst bed 240 the reducing agent can interact with material of catalyst bed 240 to remove NOx from TEG 270. TEG 270 that has been reacted with catalyst bed 240 can flow from catalyst bed 240 through second sensing panel 250. Second sensing panel 250 can be used to determine how much, if any, reducing agent remains in TEG 270.

Second sensing panel 250 can be used to determine the amount of reducing agent in TEG 270 downstream of catalyst bed 240. Second sensing panel 250 can be used to tune the amount of reducing agent injected at each grid of injection panel 200 via the automated valves. For example, the amount of reducing agent sensed by sensing zone 252B can be used to control valve 224B to, for example, prevent waste from over-injection of reducing agent or avoid incompletely treating TEG 270 by under-injection of reducing agent. As explained below, output of sensing zone 252B can additionally be used to assess the condition of catalyst bed 240 and thereby facilitate determining if there are leaks or slip conditions within exhaust duct 202.

First sensing panel 230 can be used to determine the amount of reducing agent in TEG 270 upstream of catalyst bed 240. First sensing panel 230 can be used to verify the amount of reducing agent injected at each grid of injection panel 200 via the automated valves including valve 224B. For example, the amount of reducing agent sensed by sensing zone 232B can be used to verify operation of valve 224B. For example, first sensing panel 230 can be used to determine if there is an issue with valve 224B or to determine the delta, e.g., change, in reducing agent from the upstream side of catalyst bed 240 to the downstream side of catalyst bed 240.

Furthermore, first sensing panel 230 and second sensing panel 250 can be used to diagnose potential problems with catalyst bed 240, injection panel 200 and exhaust duct 202, when working together. For example, first sensing panel 230 and second sensing panel 250 can be used to determine if excessive or limited reducing agent sensed at second sensing panel 250 is from oversaturation of catalyst bed 240, degradation of catalyst bed 240, leaking of reducing agent around catalyst bed 240, or another other causes.

Following are a list of example situations that first sensing panel 230 and second sensing panel 250 can be used to sense and assess to make adjustments to balancing valves 224B, injection panel 200 or catalyst bed 240. The list is not exhaustive of the situations that first sensing panel 230 and second sensing panel 250 can be used to assess and are provided for instruction.

If sensing zone 232B senses a high amount of reducing agent and sensing zone 252B senses a lower amount of reducing agent or no reducing agent, this can be indicative that catalyst bed 240 is functioning and/or that valve 224B is not properly adjusted. For example, a drop in reducing agent can indicate that reducing agent is being consumed by catalyst bed 240. However, a smaller than expected drop in reducing agent can also potentially mean that reducing agent is leaking around the area of interest of catalyst bed 240. Additionally, if a very low amount or no reducing agent is sensed at sensing zone 232B, this can be indicative of not enough reducing agent being provided by valve 224B. As discussed below, output of sensing zone 232B as well as output of first sensing panel 230 and second sensing panel 250 in other injection zones can be used to determine which of these cases may potentially be occurring.

If sensing zone 232B senses a high amount of reducing agent and sensing zone 252B senses a low amount of reducing agent, this can be indicative that catalyst bed 240 is functioning and valve 224B is injecting a proper amount of reducing agent because slip is low. This can be verified if other grids adjacent to grid 208B have similar results. Thus, controller 260 can take no action because operation of injection panel 200 is functioning properly. In a particular example, sensing zone 232B can sense one-hundred ppm (parts per million) of ammonia coming in and sensing zone 252B can sense only two ppm coming out, which can indicate that the corresponding part of catalyst bed 240 is very active and valve 224B is suitably tuned because the slip is low.

If sensing zone 232B senses a high amount of reducing agent and sensing zone 252B senses no reducing agent, this can be indicative that catalyst bed 240 is functioning and valve 224B is under-injecting reducing agent. This can be verified if output of other sensing zones in second sensing panel 250 are higher than output of sensing zone 252B. Thus, controller 260 can operate valve 224B to increase the amount of reducing agent being injected.

If sensing zone 232B senses a high amount of reducing agent and sensing zone 252B senses a diminished amount of reducing agent that is high, this can be indicative that catalyst bed 240 is functioning and valve 224B is injecting too much reducing agent because slip is high. This can be verified if output of other sensing zones in second sensing panel 250 are lower than output of sensing zone 252B. Thus, controller 260 can operate valve 224B to decrease the amount of reducing agent being injected.

If sensing zone 232B senses a high amount of reducing agent and sensing zone 252B senses similarly high amount of reducing agent or the same amount of reducing agent, this can be indicative that catalyst bed 240 is malfunctioning or reducing agent is passing around catalyst bed. This can be verified if output of other sensing zones in second sensing panel 250 are lower than output of sensing zone 252B. In such, scenarios it can be desirable to perform maintenance on exhaust duct 202 to check for leaks or to inspect catalyst bed 240 for degradation. In a particular example, if sensing zone 232B senses fifty ppm of ammonia coming in and sensing zone 252B also senses fifty ppm coming out, this can indicate that the corresponding part of catalyst bed 240 is malfunctioning, that there is slip of ammonia around the corresponding part of catalyst bed 240, or there is no NOx in that particular zone of the exhaust duct, which is highly unlikely.

If sensing zone 232B senses a low amount of reducing agent and sensing zone 252B senses a lower amount of reducing agent or no reducing agent, this can be indicative that catalyst bed 240 is functioning, that there is a leak within exhaust duct 202 and/or that valve 224B needs adjustment. For example, a drop in reducing agent can indicate that reducing agent is being consumed by catalyst bed 240. However, a drop in reducing agent can also potentially mean that reducing agent is leaking around the area of interest of catalyst bed 240, potentially indicating a leak within exhaust duct 202 or around catalyst bed 240. Additionally, if a very low amount or no reducing agent is sensed at sensing zone 232B, this can be indicative of not enough reducing agent being provided by valve 224B. In such scenarios, controller 260 can increase the amount of reducing agent injected by valve 224B to perform the analyses listed above. In a particular example, if all of the valves of injection panel 200 are open at 30% and all of the sensing zones of second sensing panel 250 are sensing five parts per million of ammonia, but valve 224B is open only 5% and sensing zone 252B is sensing a relatively high level of ammonia, this can indicate that that particular zone of catalyst bed 240 might be degraded, or that the ammonia is leaking around the catalyst.

If sensing zone 232B senses a low amount of reducing agent and sensing zone 252B senses the same or similar amounts of reducing agent, this can be indicative that catalyst bed 240 is malfunctioning.

FIG. 5 is a schematic cross-sectional view of sensing system 400 of the present disclosure comprising a tunable diode laser system including transmitter 402 and receiver 404. Sensing system 400 can comprise post 406, first pipe 408A, and second pipe 408B. First pipe 408A can comprise first flange 410A, first end portion 412A, second end portion 414A and internal passage 416A. Second pipe 408B can comprise second flange 410B, first end portion 412B, second end portion 414B and internal passage 416B. First pipe 408A can include first mirror 420 and second mirror 422. Second pipe 408B can include third mirror 424 and fourth mirror 425.

Transmitter 402 can emit first laser beam 426, second laser beam 428 and third laser beam 430. First laser beam 426 can comprise first stage 426A and second stage 426B. Second laser beam 428 can comprise first stage 428A, second stage 428B and third stage 428C. Third laser beam 430 can comprise first stage 430A and second stage 430B.

Transmitter 402 can include various connectors for conveying laser energy from transmitter 402 to first pipe 408A and second pipe 408B. For example, transmitter 402 can comprise first connector 432A for conveying first laser beam 426 to first flange 410A, second connector 432B for conveying second laser beam 428 to first flange 410A, and third connector 432C for conveying third laser beam 430 to second flange 410B.

Receiver 404 can include various connectors for receiving laser beams from first pipe 408A and second pipe 408B. For example, receiver 404 can comprise first connector 434A for receiving first laser beam 426 at first flange 410A, second connector 434B for receiving second laser beam 428 at second flange 410B, and third connector 434C for receiving third laser beam 430 at second flange 410B.

Transmitter 402 and receiver 404 can be connected to controller 436. Controller 436 can be connected to interface device 438 and valve 440. Controller 436 can comprise controller 260 of FIG. 4 and controller 190 of FIG. 2. Valve 440 can be representative of any or all of the valves connected to injection panel 200, such as valve 224A and valve 224B.

Transmitter 402 can comprise a tunable diode laser (TDL), which can comprise a semiconductor device similar to a light-emitting diode in which a diode is pumped directly with electrical current to produce a laser. The TDL can be used in conjunction with a laser absorption spectrometry system to measure the concentration of gaseous mixtures. TDL absorption spectroscopy can detect very low concentrations of gases, such as on the order of parts per billion (ppb) or parts per million (ppm). The laser absorption spectrometry system can comprise transmitter 402 that includes a TDL light source, transmitting optics, an optically accessible absorbing medium, receiving optics and a detector, such as receiver 404. The emission wavelength of the TDL emitted by transmitter 402 can be tuned over the characteristic absorption lines of a species, e.g., ammonia, in the gas, e.g., exhaust gas, in the path of the laser beam. This can cause a reduction of the measured signal intensity due to absorption, which can be detected by a photodiode included in receiver 404, and then used to determine the gas concentration and other properties of the exhaust gas. Controller 436 can be used to analyze the output of the TDL spectroscopy system. Interface device 438 can be used to convey information from the tunable diode laser spectroscopy system to a user, such as by presenting visual, audio and tactile information and the like. Interface device 438 can comprise a device configured to output information from sensing system 400 and enter information into sensing system 400, such as a touchscreen display or a tablet computing system. In examples, interface device 438 can provide gas concentration levels, e.g., tables or charts of numbers representative of the gas concentrations, including that of a reducing agent such as ammonia, for each of the grids of injection panel 200. In examples, interface device 438 can provide audio, visual and tactile alarms indicating that too much or too little reducing agent is being sensed. Controller 436 can additionally operate one or move instances of valve 440 to make adjustments in the amount of reducing agent levels, e.g., volumes, being injected. Interface device 438 can additionally be used to solicit feedback or confirmation from a user before changing a state of a control valve, such as by presenting a menu of options of suggested valve actions along with accept or reject options.

First pipe 408A and second pipe 408B can be mounted to exhaust duct 202 and supported thereat by post 406, which can be mounted within or outside of a wall of exhaust duct 202. For example, post 406 can be positioned alongside right panel 204D (FIG. 3) on the outside of exhaust duct 202. Thus, first flange 410A and second flange 410B can be positioned outside of exhaust duct 202 and the shafts of first pipe 408A and second pipe 408B can extend into the interior of exhaust duct 202. Second end portion 414A of first pipe 408A and second end portion 414B of second pipe 408B can be positioned within exhaust duct 202 to sense the composition of exhaust gas therebetween. In examples, sensing system 400 can be used to sense gas composition for grid 208A (FIG. 3). Thus, second end portion 414A and second end portion 414B can be positioned behind catalyst bed 240 such as at sensing zone 252A. In examples, second end portion 414A and second end portion 414B can be positioned at or near the center of grid 208A. The area between second end portion 414A and second end portion 414B can comprise sensing zone 442. Sensing zone 442 can comprise any sensing zone of first sensing panel 230, such as sensing zone 232A and sensing zone 232B, and any sensing zone of second sensing panel 250, such as sensing zone 252A and sensing zone 252B.

Second end portion 414A and second end portion 414B can be positioned at any location where it is desired to sense gas composition within exhaust duct 202. In the illustrated example, first pipe 408A and second pipe 408B are shown extending perpendicular to post 406 and thereby also perpendicular to right panel 204D, parallel to each other and having the same length. In additional examples, other configurations of insertion pipes can be used. In examples, a single insertion pipe can be used, and more than two insertion pipes can be used. Additionally, the insertion pipes can be non-parallel, can have different lengths, and can extend from post 406 at various angles.

As discussed herein, first pipe 408A and second pipe 408B can be used to locate sensing zone 442 within exhaust duct 202. First pipe 408A and second pipe 408B can also provide pathways for obtaining baseline sensor readings to compare to sensor readings in sensing zone 442. For example, first pipe 408A and second pipe 408B can be shielded from the flow of exhaust gas, but can still be filled with exhaust gas because they are not airtight. First pipe 408A and second pipe 408B can have some amount of exhaust gas and reducing agent therein that might not be representative of the exhaust gas and reducing agent where they extend to within exhaust duct 202. Thus, first laser beam 426 and third laser can be used determine the level of reducing agent in first pipe 408A and second pipe 408B, which might comprise noise added to the amount of reducing agent sensed with second laser beam 428. For example, controller 436 can determine the average amount of reducing agent along the path of each of first laser beam 426 and second laser beam 428. Thus, in order to determine the reducing agent content between second end portion 414A and second end portion 414B, it is useful to subtract the reducing agent content within each of first pipe 408A and second pipe 408B.

Transmitter 402 can emit first laser beam 426, which can travel through first connector 432A to first flange 410A. First stage 426A of first laser beam 426 can leave first connector 432A, travel through internal passage 416A, impact first mirror 420 and be reflected backward to first flange 410A as second stage 426B. Second stage 426B can enter first connector 434A for transmission to receiver 404. Controller 436 can interpret second stage 426B to determine the gas composition within first pipe 408A.

Transmitter 402 can emit third laser beam 430, which can travel through third connector 432C to second flange 410B. First stage 430A of third laser beam 430 can leave third connector 432C, travel through internal passage 416B, impact fourth mirror 425 and be reflected backward to second flange 410B as second stage 430B. Second stage 430B can enter third connector 434C for transmission to receiver 404. Controller 436 can interpret second stage 430B to determine the gas composition within second pipe 408B.

Transmitter 402 can emit second laser beam 428, which can travel through second connector 432B to first flange 410A. First stage 428A of second laser beam 428 can leave second connector 432B, travel through internal passage 416A, impact against second mirror 422 and be reflected into the interior of exhaust duct 202 as second stage 428B. Second stage 428B can travel through sensing zone 442 and impact against third mirror 424 to be reflected as third stage 428C toward second pipe 408B. Thereafter, third stage 428C can travel through internal passage 416B of second pipe 408B and enter second connector 434B for transmission to receiver 404. Controller 436 can interpret third stage 428C to determine the gas composition between first flange 410A and second flange 410B.

As such, controller 436 can be provided with three different sensor or gas composition readings. The first sensor reading can comprise the amount of reducing agent in the interior of first pipe 408A, the third sensor reading can comprise the amount of reducing agent in the interior of second pipe 408B, and the second sensor reading can comprise the total amount of reducing agent in first pipe 408A, in sensing zone 442 and in second pipe 408B. Thus, the first sensor signal and the third sensor signal can be subtracted from the second sensor signal to obtain the amount of reducing agent only within sensing zone 442.

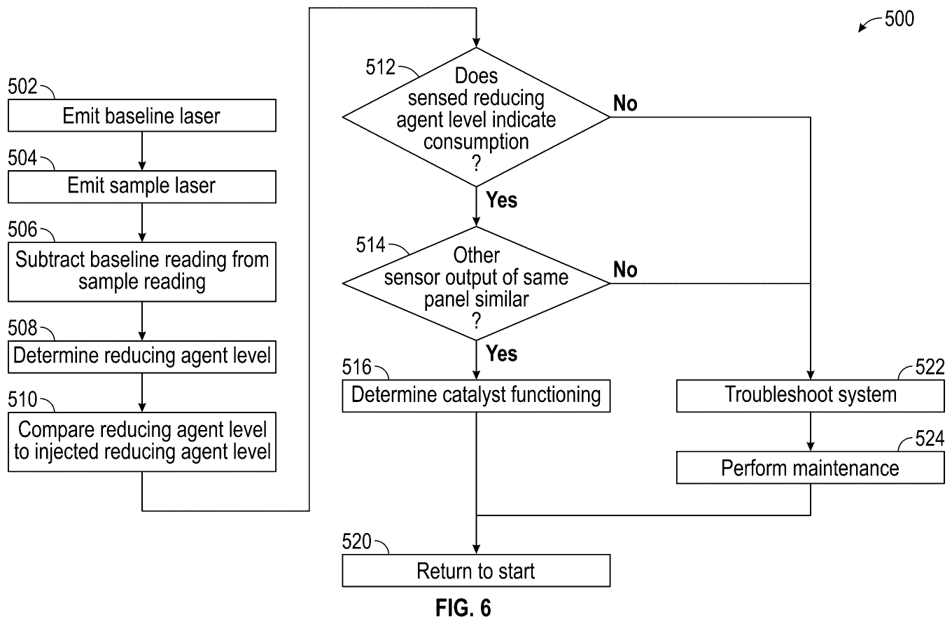

FIG. 6 is a block diagram illustrating method 500 including operation 502 through operation 530 for obtaining exhaust gas composition readings and adjusting reducing agent injection in an emissions reduction system of the present disclosure. Though discussed with reference to FIG. 1 through FIG. 5 and a particular example of an exhaust gas measurement apparatus, method 500 can encompass the use of any emissions reduction system and exhaust gas measurement apparatus consistent with the methods and systems described herein. Method 500 can additionally include fewer or greater operations other than operation 502 to operation 530. Additionally, in other examples, operation 502 through operation 530 can be performed in other sequences.

At operation 502, a first laser beam can be emitted from a transmitter into a passage or lumen within an exhaust duct. For example, first laser beam 426 can be emitted from transmitter 402 into first pipe 408A extending exhaust duct 202. First laser beam 426 can be used to determine a baseline gas composition, such as the composition of gas within first pipe 408A. Operation 502 can include emitting additional laser beams to obtain baseline gas readings in other parts of exhaust duct 202. For example, third laser beam 430 can be emitted from transmitter 402 into second pipe 408B extend from exhaust duct 202. Operation 502 can include directing first laser beam 426 and third laser beam 430 into receiver 404. For example, first mirror 420 can be used to direct first laser beam 426 back through first pipe 408A and into receiver 404, and fourth mirror 425 can be used to direct third laser beam 430 back through second pipe 408B and into receiver 404.

At operation 504, a second laser beam can be emitted from a transmitter into a passage or lumen within the exhaust duct. For example, second laser beam 428 can be emitted from transmitter 402 into first pipe 408A extending into exhaust duct 202. Operation 504 can include directing second laser beam 428 into receiver 404. In examples, second mirror 422 and third mirror 424 can be used to guide second laser beam 428 from first pipe 408A into second pipe 408B and then into second pipe 408B and on to receiver 404.

At operation 506, gas composition readings taken from first laser beam 426, second laser beam 428 and third laser beam 430. Gas composition readings from first laser beam 426 and third laser beam 430 can be subtracted from a gas composition reading of second laser beam 428. As such, precise locations within exhaust duct can be reached by extending entrance and exit pipes to the desired location to obtain baseline readings. The entrance and exit baseline pipes can be used to shield or remove noise from a sampling signal traveling to the desired location through the baseline pipes. As such, the sampling signal can be used to provide a pure measurement reading of exhaust gas composition between the baseline pipes after baseline readings from the baseline pipes are subtracted therefrom.

At operation 508, the gas composition of exhaust gas between first pipe 408A and second pipe 408B within exhaust duct 202 at sensing zone 442 can be determined. The gas composition can include a level of reducing agent, such as ammonia within exhaust gas of a gas turbine engine. In examples, the exhaust gas composition can be sensed downstream of catalyst bed 240, such as with second sensing panel 250. In examples, operation 502 and operation 504 can be repeated upstream of catalyst bed 240 with first sensing panel 230. In examples, operation 508 can be combined with operation 506. For example, the control algorithm for determine reducing agent composition can be applied to the baseline and sample readings to estimate reducing agent level in sensing zone.

At operation 510, the level of reducing agent within the exhaust gas sensed at operation 508 can be compared to levels of reducing agent introduced into exhaust duct 202, such as from injection panel 200. In examples, the level of reducing agent sensed by second sensing panel 250 can be compared to precise levels of reducing agent introduced into exhaust duct 202 as can be determined by first sensing panel 230. If catalyst bed 240 is consuming reducing agent as expected, there should be a corresponding expected drop in reducing agent between first sensing panel 230 and second sensing panel 250. In examples, the level of reducing agent sensed at second sensing panel 250 can be compared to expected levels of reducing agent based on recorded positions, e.g., percent open, of the reducing agent valves, such as valve 224A and valve 224B. In examples, the level of reducing agent sensed at second sensing panel 250 can be compared to estimated levels of reducing agent introduced into exhaust duct 202 as can generally be determined by operation of inlet valve 214 and fan 212.

At operation 512, it can be determined if the sensed level of reducing agent downstream of the catalyst bed is below the injected level, as sensed or estimated. For example, the amount that the sensed level of reducing agent is below the injected level can be compared to expected drops in reducing agent due to consumption in catalyst bed 240. As mentioned, the relative level of reducing agent can comprise a direct comparison of two sensed levels or can comprise a comparison of the sensed reducing agent level to an expected value using reducing agent valve positions.

If it is determined that the sensed level of reducing agent downstream of catalyst bed 240 is below what is understood to have been injected upstream of catalyst bed 240, e.g., according to expected consumption levels, method 500 can move to operation 514. If it is determined that the sensed level of reducing agent downstream of catalyst bed 240 is not below, e.g., not according to expected consumption levels, or above what is understood to have been injected upstream of catalyst bed 240, method 500 can move to operation 522.

At operation 514, the change in reducing agent level for the grid of injection panel 200 sensed at operation 502 and operation 504 can be compared to sensor readings for other grids of injection panel 200. For example, output of sensing zone 232A can be compared to output of sensing zone 232B. If it is determined that the reducing agent level for the grid sensed at operation 502 and operation 504 (e.g., sensing zone 232A) is similar to reducing agent levels sensed at some or all other grids (e.g., sensing zone 232B), method 500 can move to operation 516. This can be indicative that the sensed grid is acting similar to other grids, thereby indicating a reduced chance of an anomaly occurring at the sensed grid and that the select catalyst reduction system is operating properly. If it is determined that the grid sensed at operation 502 and operation 504 is not similar to what is sensed at some or all other girds, method 500 can move to operation 522. This can be indicative that the sensed grid is potentially experiencing one or more localized anomalies, or other grids are experiencing one or more anomalies. Examples of anomalies include slippage of reducing agent past the catalyst bed, over-injection of reducing agent, under-injection of reducing agent and degraded catalyst regions.

At operation 516, it can be determined that the reducing agent levels sensed at the grid of injection panel 200 at operation 502 and operation 504 indicate that catalyst bed 240 is functioning properly. As mentioned, it can be determined that the sensed grid is acting in conformity with the rest of the injection grids and the reducing agent levels being reduced are due to consumption of reducing agent in the catalyst bed. Thus, method 500 can move to operation 520 to begin the sensing process all over again.

At operation 522, troubleshooting of SCR system 154 can be performed to diagnose any issues with one or both injection panel 200 catalyst bed 240. Thereafter, maintenance can be performed at operation 534 to correct any issues or potential issues identified or determined at operation 522.

In examples, sensor readings can indicate that not as much reducing agent is being consumed compared to what is expected for a functioning catalyst bed. This can potentially be indicative of over injection of reducing agent or degradation of catalyst bed 240, or other issues.

In examples, sensor readings can indicate that a larger amount of reducing agent is being consumed compared to what is expected for a functioning catalyst bed. This can potentially be indicative of under injection of reducing agent or slippage of reducing agent around catalyst bed 240, or other issues.

In examples of operation 522, exhaust duct 202 can be inspected to evaluate or determine if leakage or slippage is occurring around catalyst bed 240. If it is determined slippage is or may be occurring, maintenance can be performed on exhaust duct 202 or catalyst bed 240 to seal leaks or reroute slippage at operation 524.

In examples of operation 522, catalyst bed 240 can be inspected for damage or degradation to account for reducing agent not being consumed. If it is determined that catalyst bed 240 is degraded at operation 522, catalyst bed 240 can be replaced if it is determined that it is greatly degraded at operation 524. In examples, catalyst bed 240 can be replaced if it is less than seventy-five percent effective, less than fifty percent effective or less than twenty-five percent effective.

In examples of operation 522, injection panel 200 can be inspected to determine if valves are set to release a proper or desired amount of reducing agent. If it is determined that the valves are releasing too much reducing agent, the valves can be adjusted to more closed positions to prevent over-injection of reducing agent. If it is determined that the valves are releasing too little reducing agent, the valves can be adjusted to more opened positions to prevent under-injection of reducing agent. Controller 260 and controller 436 can be used to perform the valve adjustments.

TABLE 1

| | Reducing Agent | Reducing Agent @ | |
|---|---|---|---|
| | @ Sensed Grid | Other Sensed Grid | |
| Case | Location | Locations | Possible Catalyst Bed Analysis |
| 1 | Expected drop | Expected drop | Functioning (operation 516) |
| 2 | Expected drop | Larger than expected | Slippage and/or under injection at |
| | | drop | other parts of catalyst bed |
| 3 | Expected Drop | Smaller than expected | Over injection and/or catalyst |
| | | drop/No drop | degradation at other parts of catalyst |
| | | | bed |
| 4 | Larger than | Larger than expected | Global slippage and/or under injection |
| | expected drop | drop | throughout catalyst bed |
| 5 | Larger than | Expected drop | Localized slippage and/or under |
| | expected drop | | injection at sensed location |
| 6 | Smaller than | Smaller than expected | Global over injection and/or catalyst |
| | expected drop | drop | degradation throughout catalyst bed |
| 7 | Smaller than | Expected drop | Localized over injection and/or |
| | expected drop | | catalyst degradation at sensed location |

Examples trouble shooting scenarios of the present disclosure are summarized in Table 1. However, other troubleshooting actions and maintenance actions can be performed. For example, in some scenarios, if the sensed reducing agent levels do not meet or are not close to expected values at any location of the catalyst bed multiple troubleshooting and maintenance operations can be performed regardless of what is listed in Table 1.

At operation 516 or operation 524, method 500 can return to operation 502 to continue to monitor operation of emissions reduction system 16 (FIG. 1) or SCR system 154 (FIG. 2).

This present disclosure provides a real-time sensing and control system for selective catalytic reduction systems used in gas turbine power plants. The system can use tunable diode laser technology to measure gas composition at multiple points within an exhaust duct, enabling precise control of ammonia injection for emissions reduction. The present disclosure provides a plurality of benefits over prior art systems and methods.

Real-time measurement and feedback: The present disclosure facilitates rapid sensing of gas composition compared to traditional extractive methods and allows for continuous monitoring and adjustment of ammonia injection.

Improved emissions control: The present disclosure allows for more precise control of ammonia injection based on actual measured conditions, thereby reducing ammonia slip and waste and helping to maintain compliance with emissions regulations.

Enhanced operational efficiency: The present disclosure comprises an automated valve control system that can adjusts ammonia flow in real-time and can thus detect catalyst degradation and system issues early, thereby extending catalyst life through optimized or improved operation and reducing operational costs through better reagent management.

Better diagnostic capabilities: The present disclosure provides spatial mapping of gas composition across the exhaust duct, thereby facilitating detection of catalyst degradation patterns, helping to identify leaks or bypass conditions, and allowing troubleshooting of mechanical issues.

Operational flexibility: The present disclosure allows for emissions reduction systems to adjust to different operating conditions of a gas turbine engine automatically, thereby handling varying load conditions and fuel types and maintaining optimal or improved performance across different operating states.

The present disclosure presents a significant improvement over conventional systems that rely on manual valve adjustment and extractive sampling methods, offering both improved environmental performance and operational benefits.

### VARIOUS NOTES & EXAMPLES

Example 1 is a power production system comprising: a gas turbine engine configured to combust a fuel to produce a gas that can be used to produce rotational shaft power for generating electricity; an exhaust duct configured to receive the gas from the gas turbine engine, the exhaust duct having a cross-sectional area; one or more catalyst panels positioned in the exhaust duct to substantially cover the cross-sectional area and interact with the gas; an injection system configured to release a reductant into the gas upstream of the one or more catalyst panels, the injection system comprising a plurality of grids, each grid of the plurality of grids configured to release reductant over a portion of the cross-sectional area; and a sensing system positioned in the exhaust duct to sense composition of gas within the exhaust duct, the sensing system comprising: a first plurality of gas composition sensors positioned in the exhaust duct, wherein each of the first plurality of gas composition sensors is paired with one of the plurality of grids of the injection system to provide a gas composition reading for each of the plurality of grids.

In Example 2, the subject matter of Example 1 optionally includes wherein the first plurality of gas composition sensors is positioned downstream of the one or more catalyst panels.

In Example 3, the subject matter of Example 2 optionally includes a second plurality of gas composition sensors positioned between the injection system and the one or more catalyst panels.

In Example 4, the subject matter of any one or more of Examples 1-3 optionally include a plurality of electronically controllable valves connected to the plurality of grids in a one-to-one relationship.

In Example 5, the subject matter of Example 4 optionally includes a controller configured to adjust each of the plurality of electronically controllable valves based on output of the first plurality of gas composition sensors.

In Example 6, the subject matter of any one or more of Examples 1-5 optionally include wherein each of the first plurality of gas composition sensors comprises a tunable diode laser.

In Example 7, the subject matter of Example 6 optionally includes wherein each of the first plurality of gas composition sensors comprises a plurality of tunable diode lasers configured to generate at least one baseline sensor reading and a sample sensor reading.

In Example 8, the subject matter of Example 7 optionally includes wherein the sensing system comprises one or more insertion pipes to guide lasers of the plurality of tunable diode lasers to locations within the exhaust duct.

In Example 9, the subject matter of Example 8 optionally includes wherein the one or more insertion pipes comprises a first insertion pipe and a second insertion pipe, wherein a space between distal ends of the first insertion pipe and the second insertion pipe within the exhaust duct comprise a sensing zone.

In Example 10, the subject matter of Example 9 optionally includes a controller configured to determine gas composition readings from the first plurality of gas composition sensors, wherein the controller is configured to subtract readings within the first pipe and the second pipe from a reading extending through the first pipe and the second pipe to determine gas composition within the sensing zone.

Example 11 is a gas composition sensor for use in an exhaust duct of a gas turbine power plant, the gas composition sensor comprising: a first pipe comprising: a first end portion connected to the exhaust duct; and a second end portion positioned within the exhaust duct at a location where gas composition is to be sensed; a transmitter configured to emit: a first laser beam to sense a first gas composition within the first pipe; and a second laser beam to sense gas composition in the exhaust duct outside of the second end portion; a receiver configured to receive the first laser beam and the second laser beam to determine a first composition signal and a second composition signal; and a controller configured to subtract the first composition signal from the second composition signal to determine gas composition at the location.

In Example 12, the subject matter of Example 11 optionally includes a first mirror positioned at the second end portion of the first pipe to reflect the first laser beam back through the first pipe to the receiver.

In Example 13, the subject matter of Example 12 optionally includes a second pipe comprising: a third end portion connected to the exhaust duct; and a fourth end portion positioned within the exhaust duct spaced from the second end portion; and a second mirror positioned at the fourth end portion of the second pipe; wherein: the transmitter is configured to emit a third laser beam into the second pipe to sense gas composition within the second pipe; and the second mirror is configured to reflect the third laser beam back through the second pipe to the receiver.

In Example 14, the subject matter of Example 13 optionally includes a third mirror positioned at the second end portion of the first pipe to reflect the first laser beam toward the fourth end portion of the second pipe; and a fourth mirror positioned at the fourth end portion of the second pipe to receive the first laser beam from the third mirror and reflect the first laser beam through the second pipe to the receiver.

In Example 15, the subject matter of Example 14 optionally includes a first plurality of connections to connect the first end portion and the third end portion to the transmitter; and a second plurality of connections to connect the first end portion and the third end portion to the receiver.

In Example 16, the subject matter of any one or more of Examples 11-15 optionally include wherein the transmitter and the receiver comprise portions of a tunable diode laser absorption spectroscopy system.

Example 17 is a method of determining composition of exhaust gas in a gas turbine power plant having an emissions reduction system configured to dispense reductant onto a catalyst bed, the method comprising: emitting a first laser beam into a first sensing tube extending into an exhaust duct; emitting a second laser beam into the first sensing tube and into the exhaust duct; sensing a first reductant level in the first sensing tube using the first laser beam; sensing a second reductant level in the exhaust duct using the second laser beam; subtracting the first reductant level from the second reductant level to determine a first reductant value; performing a comparison of the first reductant value to an expected reductant value; and adjusting a reductant valve to adjust an amount of reductant dispensed onto the catalyst bed based on the comparison.

In Example 18, the subject matter of Example 17 optionally includes wherein: performing the comparison of the first reductant value to the expected reductant value comprises determining the first reductant value is above the expected reductant value; and adjusting the reductant valve to adjust the amount of reductant dispensed onto the catalyst bed comprises reducing flow of reductant into the exhaust duct.

In Example 19, the subject matter of any one or more of Examples 17-18 optionally include wherein: performing the comparison of the first reductant value to the expected reductant value comprises determining the first reductant value is below the expected reductant value; and adjusting the reductant valve to adjust the amount of reductant dispensed onto the catalyst bed comprises increasing flow of reductant into the exhaust duct.

In Example 20, the subject matter of any one or more of Examples 17-19 optionally include wherein sensing the first reductant level in the first sensing tube using the first laser beam and sensing the second reductant level in the exhaust duct using the second laser beam comprises measuring gas composition using one of more tunable diode laser sensors.

In Example 21, the subject matter of any one or more of Examples 17-20 optionally include sensing gas composition upstream of the catalyst bed to determine catalyst degradation patterns by comparing upstream and downstream measurements.

In Example 22, the subject matter of any one or more of Examples 17-21 optionally include wherein adjusting the reductant valve to adjust the amount of reductant dispensed onto the catalyst bed comprises: continuously adjusting reductant flow to each section of an ammonia distribution grid based on changing operating conditions of the gas turbine power plant.

In Example 23, the subject matter of any one or more of Examples 17-22 optionally include sensing a third reductant level in a second sensing tube using a third laser beam; and subtracting the first reductant level and the third reductant level from the second reductant level to determine the first reductant value.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A power production system comprising:
a gas turbine engine configured to combust a fuel to produce a gas that can be used to produce rotational shaft power for generating electricity;
an exhaust duct configured to receive the gas from the gas turbine engine, the exhaust duct having a cross-sectional area;
one or more catalyst panels positioned in the exhaust duct to substantially cover the cross-sectional area and interact with the gas;
an injection system configured to release a reductant into the gas upstream of the one or more catalyst panels, the injection system comprising a plurality of grids, each grid of the plurality of grids configured to release reductant over a portion of the cross-sectional area; and
a sensing system positioned in the exhaust duct to sense composition of gas within the exhaust duct, the sensing system comprising:
a first plurality of gas composition sensors positioned in the exhaust duct, wherein each of the first plurality of gas composition sensors is paired with one of the plurality of grids of the injection system to provide a gas composition reading for each of the plurality of grids.

2. The power production system of claim 1, wherein the first plurality of gas composition sensors is positioned downstream of the one or more catalyst panels.

3. The power production system of claim 2, further comprising a second plurality of gas composition sensors positioned between the injection system and the one or more catalyst panels.

4. The power production system of claim 1, further comprising a plurality of electronically controllable valves connected to the plurality of grids in a one-to-one relationship.

5. The power production system of claim 4, further comprising a controller configured to adjust each of the plurality of electronically controllable valves based on output of the first plurality of gas composition sensors.

6. The power production system of claim 1, wherein each of the first plurality of gas composition sensors comprises a tunable diode laser.

7. The power production system of claim 6, wherein each of the first plurality of gas composition sensors comprises a plurality of tunable diode lasers configured to generate at least one baseline sensor reading and a sample sensor reading.

8. The power production system of claim 7, wherein the sensing system comprises one or more insertion pipes to guide lasers of the plurality of tunable diode lasers to locations within the exhaust duct.

9. The power production system of claim 8, wherein the one or more insertion pipes comprises a first insertion pipe and a second insertion pipe, wherein a space between distal ends of the first insertion pipe and the second insertion pipe within the exhaust duct comprise a sensing zone.

10. The power production system of claim 9, further comprising a controller configured to determine gas composition readings from the first plurality of gas composition sensors, wherein the controller is configured to subtract readings within the first pipe and the second pipe from a reading extending through the first pipe and the second pipe to determine gas composition within the sensing zone.

11. A gas composition sensor for use in an exhaust duct of a gas turbine power plant, the gas composition sensor comprising:
a first pipe comprising:
a first end portion connected to the exhaust duct; and
a second end portion positioned within the exhaust duct at a location where gas composition is to be sensed;
a transmitter configured to emit:
a first laser beam to sense a first gas composition within the first pipe; and
a second laser beam to sense gas composition in the exhaust duct outside of the second end portion;
a receiver configured to receive the first laser beam and the second laser beam to determine a first composition signal and a second composition signal; and
a controller configured to subtract the first composition signal from the second composition signal to determine gas composition at the location.

12. The gas composition sensor of claim 11, further comprising:
a first mirror positioned at the second end portion of the first pipe to reflect the first laser beam back through the first pipe to the receiver.

13. The gas composition sensor of claim 12, further comprising:
a second pipe comprising:
a third end portion connected to the exhaust duct; and
a fourth end portion positioned within the exhaust duct spaced from the second end portion; and
a second mirror positioned at the fourth end portion of the second pipe;
wherein:
the transmitter is configured to emit a third laser beam into the second pipe to sense gas composition within the second pipe; and
the second mirror is configured to reflect the third laser beam back through the second pipe to the receiver.

14. The gas composition sensor of claim 13, further comprising:
a third mirror positioned at the second end portion of the first pipe to reflect the first laser beam toward the fourth end portion of the second pipe; and
a fourth mirror positioned at the fourth end portion of the second pipe to receive the first laser beam from the third mirror and reflect the first laser beam through the second pipe to the receiver.

15. The gas composition sensor of claim 14, further comprising:
a first plurality of connections to connect the first end portion and the third end portion to the transmitter; and
a second plurality of connections to connect the first end portion and the third end portion to the receiver.

16. The gas composition sensor of claim 11, wherein the transmitter and the receiver comprise portions of a tunable diode laser absorption spectroscopy system.

17. A method of determining composition of exhaust gas in a gas turbine power plant having an emissions reduction system configured to dispense reductant onto a catalyst bed, the method comprising:
emitting a first laser beam into a first sensing tube extending into an exhaust duct;
emitting a second laser beam into the first sensing tube and into the exhaust duct;
sensing a first reductant level in the first sensing tube using the first laser beam;
sensing a second reductant level in the exhaust duct using the second laser beam;
subtracting the first reductant level from the second reductant level to determine a first reductant value;
performing a comparison of the first reductant value to an expected reductant value; and
adjusting a reductant valve to adjust an amount of reductant dispensed onto the catalyst bed based on the comparison.

18. The method of claim 17, wherein:
performing the comparison of the first reductant value to the expected reductant value comprises determining the first reductant value is above the expected reductant value; and
adjusting the reductant valve to adjust the amount of reductant dispensed onto the catalyst bed comprises reducing flow of reductant into the exhaust duct.

19. The method of claim 17, wherein:
performing the comparison of the first reductant value to the expected reductant value comprises determining the first reductant value is below the expected reductant value; and
adjusting the reductant valve to adjust the amount of reductant dispensed onto the catalyst bed comprises increasing flow of reductant into the exhaust duct.

20. The method of claim 17, wherein sensing the first reductant level in the first sensing tube using the first laser beam and sensing the second reductant level in the exhaust duct using the second laser beam comprises measuring gas composition using one of more tunable diode laser sensors.

21. The method of claim 17, further comprising sensing gas composition upstream of the catalyst bed to determine catalyst degradation patterns by comparing upstream and downstream measurements.

22. The method of claim 17, wherein adjusting the reductant valve to adjust the amount of reductant dispensed onto the catalyst bed comprises: continuously adjusting reductant flow to each section of an ammonia distribution grid based on changing operating conditions of the gas turbine power plant.

23. The method of claim 17, further comprising:
sensing a third reductant level in a second sensing tube using a third laser beam; and
subtracting the first reductant level and the third reductant level from the second reductant level to determine the first reductant value.
